(19) United States
(12) Patent Application Publication
(10) Pub. No.: US 2003/0129255 A1
(43) Pub. Date: Jul. 10, 2003

(54) Colloidal silver suspension for treating skin problems in tropical and decorative fish

(76) Inventors: Roy E. Underwood, Young Harris, GA (US); Mark A. Underwood, Young Harris, GA (US)

Correspondence Address: Harry I. Leon, 924 Bowen St., N.W., Atlanta, GA, 30318, US

(21) Appl. No.: 10/336,065

(22) Filed: Jan. 3, 2003

Related U.S. Application Data
(60) Provisional application No. 60/344,624, filed on Jan. 4, 2002.

(51) Int. Cl. A61K 33/38

(52) U.S. Cl. 424/618

(57) ABSTRACT

This is a method of treating a wide range of skin lesions in tropical and decorative fish in which the animals to be treated are exposed to dilute suspensions of colloidal silver. During the course of treatment, colloidal silver is added periodically to the aquarium in which the fish lives. so that the water therein has a silver concentration on the order of 1 ppb. A suitable treatment procedure entails adding 1 teaspoon of colloidal silver which contains about 6 ppm of silver to 10 gallons of aquarium water every other day. This treatment has been found effective in healing some fish, ever those with rather large wounds in a matter of days. If a longer treatment period is required, then the colloidal silver addition is continued at the same rate and then 50 percent of the water is changed once a week, until the lesion(s) have healed. It was also found that bacteria and virus skin problems could be prevented by having silver colloidal concentrations of 0.3-0.5 ppb in the aquarium water.

### RELATED U.S. APPLICATION DATA

This application takes advantage of Provisional Patent having the same title and inventors filed Jan. 4. 2002, and having Ser. No. 60,344,624

### BACKGROUND OF THE INVENTION

The presence of skin lesions in tropical and decorative fish is commonplace. Whether caused by scrapes during handling or a skin disease, these ailments must be treated with great care. Otherwise, because of the extreme sensitivity of these fish, they may suffer a life-threatening upset, possibly as a result of an allergic reaction triggered by exposure to certain chemicals, and succumb. While conceivable treatment scenarios include direct injection as well as the administration of a liquid medicine orally or in powdered form in the fish feed, the easiest method, for a large number of fish, is to add therapeutic substance(s) directly to their aquarium tank water.

### SUMMARY OF THE INVENTION

Tests have shown that skin problems in tropical and decorative fish can be treated successfully by adding a highly dilute suspension of colloidal silver to the aquarium water in which such fish are living. Among the species which have been treated are koi, goldfish and a variety of tropical and marine fish, which collectively exhibited a wide range of skin lesions. These lesions include ulcer diseases and fin and mouth rot all of which are bacteria eating holes in the fish. There is also help given on treatment of skin virus problems such as herpes.

In accordance with the present invention, a method of treating skin problems in fish comprises adding a teaspoon (5 ml) of a suspension of colloidal silver in which the concentration of silver ranges between 6 ppm and 12 ppm (parts per million) to 10 gallons of water in an aquarium every other day and then changing 50% of the water once a week, so that the water to which the fish to be treated are exposed has a silver concentration initially in the range of 0.77 ppb to 1.5 ppb (parts per billion). Within a matter of days this treatment has been found effective in healing some fish, even those with rather large wounds. Further, since the effective concentration of silver is so dilute that some nitrifying bacteria can survive this treatment, so some bio-filtration is still possible.

### TYPICAL TEST PROCEDURE AND RESULTS

A typical test using koi fish which initially suffered from a large skin ulcer of about one inch long and a about inch wide. This lesion was observed to have been completely healed after the fish was exposed, for a period of 29 days, to an aqueous solution containing initially 1.3 ppb of colloidal silver. A cup of Zeolite was also added to the 10 gallon aquarium housing this fish to reduce ammonia buildup. Further, the fish was fed every other day 2 pellets per 6 inch length of fish with Ichiban Koi Food, a product of Misty Mountain, to help the animal maintain an active immune system.

Some fish exposed to the same concentration of colloidal silver were found to heal completely in less than two weeks.

Test were conducted with multiple fish in the 10 gallon test and it was found that good results if the total length of the combination of the fish (measured nose to tail fin) was 16 inches in length or less.

Fish exposed to colloidal silver at concentrations as high as 10 ppb were shown to have no toxic reaction. The lethal dose concentration of colloidal silver has not been established at this time.

It was also found that skin problems could be prevented from occurring in tropical and decorative fish by having silver colloidal concentration in the aquarium of about 0,5 ppb.

### METHOD OF PREPARING THE COLLOIDAL SILVER SUSPENSION

A suitable colloidal silver suspension was prepared, using electrolysis, by applying an electric current which varied between 9 and 33 volts d.c. to two silver plates placed in a vat of distilled water. The concentration of silver in this suspension was determined analytically to lie in the range of between 6 ppm an 12 ppm. The stock solution is 10 ppm is now available at the inventors' corporation, Misty Mountain.

It is understood that those skilled in the art may conceive other applications, modifications and/or changes in the invention described above. Any such applications, modifications or changes which fall within the purview of the description are intended to be illustrative and not intended to be limitative. The scope of the invention is limited only by the scope of the claims appended hereto:

It is claimed:

1. A method of treating skin problems in tropical and decorative fish comprising:
(a) an addition of a therapeutic substance(s) directly to their aquarian tank water;
(b) the therapeutic solution is the addition of a silver colloidal solution in the order of 1 part per billion.

2. A method of treating skin problems in tropical and decorative fish comprising the following added to a ten gallon aquarium according to claim 1 which further comprises of an addition of eight ounce of Zeolite.

3. The method according to claim 1 which further comprises changing about 50 percent of the aquarium water once a week, during the course if treatment.

4. A method of treating skin problems in tropical and decorative fish comprising the following added to a ten gallon aquarium:
(a) an addition of tablespoon (5 ml) of a suspension of colloidal sliver in which the concentration of silver ranges between 6 ppm and 12 ppm (parts per million);
(b) an eight ounce addition of Zeolite;
(c) a 2 pellets Ichiban Koi Food per 6 inch length of fish every other day

5. A method of treating bacteria and virus skin infections in tropical and decorative fish comprising using a concentration of silver in an aqueous solution in the range of 0.3 ppb to 10 ppb in the aquarium water.
